(19) United States
(12) Patent Application Publication
(10) Pub. No.: US 2026/0334475 A1
(43) Pub. Date: Oct. 29, 2026

(54) NANOZYME AND PREPARATION METHOD THEREOF, AND AGRICULTURAL FORMULATION AND USE THEREOF

(71) Applicant: Anhui Agricultural University, Hefei City (CN)

(72) Inventors: Haiqun CAO, Hefei City (CN); Lihong ZHANG, Hefei City (CN); Min LIAO, Hefei City (CN); Quan GAO, Hefei City (CN); Chunlin YANG, Hefei City (CN)

(21) Appl. No.: 19/418,152

(22) Filed: Dec. 12, 2025

(30) Foreign Application Priority Data
Apr. 24, 2025 (CN) 202510519378.0

(51) Int. Cl. A01N 59/24; A01N 43/36; A01N 59/08; A01N 59/20; A01P 3/00; A01P 21/00; B82Y 5/00; B82Y 30/00; B82Y 40/00

(52) U.S. Cl. CPC A01N 59/24; A01N 43/36; A01N 59/08; A01N 59/20; A01P 3/00; A01P 21/00; B82Y 5/00; B82Y 30/00; B82Y 40/00

(57) ABSTRACT

A nanozyme and a preparation method thereof, and an agricultural formulation and use thereof are provided. The nanozyme, prepared from raw materials including, in parts by weight: 1 part to 6 parts of a metal salt, 0.5 parts to 4 parts of a surfactant, and 1 part to 1.04 parts of an acidic reagent; where the metal salt includes one or more selected from the group consisting of an iron salt, a manganese salt, a calcium salt, a zinc salt, and a copper salt; and a metal-organic framework (MOF) material of the nanozyme is Prussian-blue.

### CROSS REFERENCE TO RELATED APPLICATION

This patent application claims the benefit and priority of Chinese Patent Application No. 202510519378.0 filed with the China National Intellectual Property Administration on Apr. 24, 2025, and entitled with “NANOZYME AND PREPARATION METHOD THEREOF, AND AGRICULTURAL FORMULATION AND USE THEREOF”, the disclosure of which is incorporated by reference herein in its entirety as part of the present application.

### TECHNICAL FIELD

The present disclosure relates to the technical field of nanomaterials, and in particular to a nanozyme and a preparation method thereof, and an agricultural formulation and use thereof.

### BACKGROUND

Nanozymes, as an environmentally friendly catalytic material, have a size range on the nanometer scale and exhibit enzyme-like reaction kinetics and specificity similar to natural enzymes. Nanozymes have broad applications in fields such as biology and medicine. In the agricultural field, nanozymes have strong environmental resistance and micro-fertilizer effects, making them particularly suitable for large-scale industrial production. However, despite the promising application prospects of nanozymes in the agriculture field, the integrated technology of pesticides and fertilizers based on nanozymes has not yet been widely adopted.

Seed coating agents are widely used in agricultural production. The seed coating agents provide essential nutrition and effectively prevent and control pests and diseases by coating seeds, thereby playing a critical role in promoting the healthy growth of crops and increasing agricultural yield. However, traditional seed coating agents generally contain high concentrations of pesticides, which can easily cause phytotoxicity to seeds, affecting their germination rate and seedling growth. Additionally, traditional seed coating agents have a short duration of efficacy and low pesticide utilization efficiency, and may introduce toxic warning dyes. This not only adversely affects the ecological environment but also fails to meet the demands of green and sustainable agricultural development.

Therefore, there is an urgent need for products and technologies that can address the issues of phytotoxicity and abiotic stress caused by traditional seed coating agents, improve the effective utilization of pesticides, and promote the healthy growth of seeds.

### SUMMARY

Based on this, the present disclosure provides a nanozyme that can effectively degrade free radicals produced under pesticide stress, reducing their toxic effects on biological systems. When used as a seed coating material, the nanozyme not only can lower the risk of phytotoxicity to seeds from traditional pesticides but also significantly improve the germination rate of seeds and the growth quality of seedlings. Correspondingly, a nanozyme and a preparation method thereof, and an agricultural formulation and use thereof are provided.

The specific technical solutions are as follows:

A first aspect of the present disclosure is to provide a nanozyme, prepared from raw materials including, in parts by weight:
1 part to 6 parts of a metal salt;
0.5 parts to 4 parts of a surfactant; and
1 part to 1.04 parts of an acidic reagent; wherein
the metal salt comprises one or more selected from the group consisting of an iron salt, a manganese salt, a calcium salt, a zinc salt, and a copper salt; and
a metal-organic framework (MOF) material of the nanozyme is Prussian-blue.

In some embodiments, the surfactant includes a tea seed cake extract; and
the acidic reagent includes citric acid.

Another aspect of the present disclosure is to provide a method for preparing the nanozyme, including:
mixing the raw materials for preparing the nanozyme, and subjecting a resulting mixture to a reaction and then drying to obtain the nanozyme.

In some embodiments, the mixing is conducted at a stirring speed of 500 r/min to 700 r/min for 10 min to 30 min;
the reaction is conducted at a temperature of 60 °C to 80 °C for 24 h to 48 h; and
the drying is conducted by a mode including one or more selected from the group consisting of vacuum drying and freeze drying.

Another aspect of the present disclosure is to provide an agricultural formulation, including: the nanozyme.

In some embodiments, the agricultural formulation includes a seed coating agent; where
the nanozyme accounts for 5% to 9% based on a weight of the seed coating agent; and where
the seed coating agent includes following components, in parts by weight:
2 parts to 10 parts of a pesticide;
5 parts to 9 parts of the nanozyme;
1.5 parts to 3 parts of a thickener;
0.75 parts to 1.5 parts of a biobased polymer;
0.1 parts to 0.5 parts of an organic acid;
3 parts to 5 parts of an organic nitride;
0.5 parts to 1.5 parts of a mineral material; and
0.1 parts to 0.3 parts of a film-forming agent.

In some embodiments, the organic acid includes one or more selected from the group consisting of salicylic acid, citric acid, ascorbic acid, and tartaric acid.

In some embodiments, the mineral material includes one or more selected from the group consisting of attapulgite, diatomite, bentonite, and sepiolite.

Another aspect of the present disclosure is to provide use of the agricultural formulation in improving a germination rate of a seed and a growth quality of a seedling.

In some embodiments, the seed is derived from one or more selected from the group consisting of soybean, wheat, corn, rapeseed, peanut, cotton, cucumber, tomato, and carrot.

In the present disclosure, the raw materials for preparing the nanozyme include specific components with specific proportional relationships, where the metal salt provides an active center and a structural foundation of the nanozyme, that is, the presence of the metal ions can provide the active center, endowing the nanozyme with a catalytic activity, similar to the catalytic effect of the metal ions at an active site in natural enzymes; the surfactant controls the size and dispersity of the nanozyme; and the acidic reagent regulates reaction conditions. The coordination among these three raw materials provides a guarantee for the successful preparation and performance optimization of the nanozyme. Through the synergistic interaction of the components in their specific proportional relationships, a surfactant-modified nanomaterial exhibits desirable water solubility and can stably exist in the form of a high-concentration colloid. When being subsequently applied in seedling cultivation, the nanomaterial has the effects of promoting growth, enhancing stress resistance, increasing yield, and improving quality of crops. Additionally, the nanozyme itself possesses a bright blue color, and thus it can replace the highly toxic organic dyes commonly used in commercial seed coating agents in a green way.

### BRIEF DESCRIPTION OF THE DRAWINGS

FIG. 1 shows an optical microscope image of the agricultural formulation in Example 1.

FIG. 2 shows a photograph of the agricultural formulation in Example 1.

FIG. 3 shows a phenotypic image of the agricultural formulation in Example 1.

FIG. 4 shows a histogram of the effect of the agricultural formulation in Example 1 on soybean germination rate.

FIG. 5 shows an effect diagram of the agricultural formulation in Example 1 on soybean germination status.

FIG. 6 shows a histogram of the effect of the agricultural formulation in Example 1 on soybean sprout length.

FIG. 7 shows a photograph of the promotion of soybean growth by the agricultural formulation in Example 1.

FIG. 8 shows the effect of the agricultural formulation in Example 1 on soybean plant height.

FIG. 9 shows the effect of the agricultural formulation in Example 1 on soybean root length.

FIG. 10 shows the effect of the agricultural formulation in Example 1 on the fresh weight of soybean plants.

FIG. 11 shows a photograph of the effect of the agricultural formulation in Example 1 on salt stress resistance in soybean plants.

FIG. 12 shows a histogram of chlorophyll content in soybean plants after salt stress under treatment with the agricultural formulation in Example 1.

### DETAILED DESCRIPTION OF THE EMBODIMENTS

To facilitate the understanding of the present disclosure, the present disclosure will be described more comprehensively below, and the preferred embodiments of the present disclosure are given as follows. However, the present disclosure may be realized in various forms without being limited to the embodiments described herein. On the contrary, these embodiments are provided to make the present disclosure more thorough and comprehensive.

The implementation of the present disclosure will be described below in detail with some embodiments and examples. The embodiments are implemented on the premise of the technical solutions of the present disclosure, and the detailed embodiments and specific operation processes are given. The scope of the present disclosure, however, is not limited to the following embodiments.

Unless otherwise defined, all technical and scientific terms used herein have the same meaning as commonly understood by those skilled in the technical field of the present disclosure. The terms used in the specification of the present disclosure are merely for the purpose of describing specific embodiments, rather than to limit the present disclosure.

### TERM

Unless otherwise stated or where there is a contradiction, the terms or phrases used herein have the following meanings:

In the present disclosure, terms, such as “a plurality of” or “multiple”, unless otherwise specified, mean a quantity greater than 2 or equal to 2. For example, “one or more” means one or two or more.

In the present disclosure, terms “further” or “particularly” are used for descriptive purposes and indicate differences in content, but should not be understood as limiting the scope of the present disclosure.

In the present disclosure, the technical features described in an open way include a closed technical solution composed of the listed features, and also include an open technical solution including the listed features.

In the present disclosure, when a numerical interval (i.e., a numerical range) is involved, unless otherwise specified, the distribution of optional numerical values within the numerical interval is considered continuous and includes the two endpoints of the numerical interval (i.e., the minimum and maximum values), as well as every numerical value between these two endpoints. Unless otherwise specified, when the numerical interval refers only to integers within that interval, it includes the two integer endpoints and every integer between them, equivalent to explicitly listing each integer. When multiple numerical ranges are provided to describe a feature or characteristic, these numerical ranges may be combined. In other words, unless otherwise indicated, the numerical ranges disclosed herein should be interpreted as including any and all subranges falling within them. The “numerical value” in the numerical interval may be any quantitative value, such as a number, percentage, ratio, etc. “Numerical interval” is broadly defined to include percentage intervals, ratio intervals, proportion intervals, and other types of numerical intervals.

In the present disclosure, unless otherwise specified, temperature parameters may be either constant temperature treatments or variable within a certain temperature range. It should be understood that “constant temperature treatment” allows the temperature to fluctuate within the precision control range of the instrument. The temperature is allowed to fluctuate within the range of, for example, ±5 °C, ±4 °C, ±3 °C, ±2 °C, or ±1 °C.

In the present disclosure, for units associated with data ranges, if the unit is provided only after the right endpoint, it indicates that the units of the left and right endpoints are the same. For example, “2-5 h” means that the unit of both the left endpoint “2” and the right endpoint “5” is h (hour).

As an environmentally friendly catalytic material, nanozymes have a size range on the nanometer scale and exhibit kinetic characteristics and specificity of enzymatic reaction similar to natural enzymes. They have broad applications in fields such as biology and medicine. In the agricultural field, through integrated pesticide-fertilizer seed coating technology, nanozymes are expected to address the issues of phytotoxicity and abiotic stress caused by traditional seed coating agents, improve the effective utilization of pesticides, and promote the healthy growth of seeds. First, nanozymes can significantly enhance the nutrient absorption efficiency of plant roots. Applying nanozymes to plant roots can improve the ability of plants to absorb nutrient elements from the soil, thereby increasing the growth speed and yield of plants. Second, nanozymes improve the resistance of seeds to pesticides by catalyzing active oxygen, thereby alleviating the degree of phytotoxicity of seeds. Additionally, nanozymes are helpful to improve the stress resistance of plants. When plants face environmental stresses such as drought, extreme temperatures, salinity, alkalinity, and heavy metals, they are susceptible to damage. Nanozymes enhance the stress resistance of plants by regulating the metabolic pathways and signal transduction pathways, enabling plants to better adapt to harsh environmental conditions. The potential application of nanozymes in promoting crop growth, development, and increasing yield brings new breakthroughs for high-quality agricultural production. More importantly, nanozymes possess multiple advantages, such as simple preparation processes, stable chemical properties, desirable repeatability of catalytic reactions, strong environmental resistance, and micro-fertilizer effects, making them particularly suitable for large-scale industrial production.

Furthermore, the seed coating agents utilize film-forming materials such as biobased polymers and thickeners to form a “biofilm hydrogel” with a three-dimensional network structure through cross-linking and self-assembly with organic acids. This biofilm is highly sensitive to changes in the external environment and can respond to environmental stimuli (e.g., pH, temperature, humidity, and salinity) through the swelling and contraction of volume, thereby intelligently controlling the release rate of pesticides and fertilizers, improving their utilization efficiency, while also having the effects of moisture conservation and soil quality improvement. However, the distinctive features of nano-seed coating agents and their preparation methods in traditional technology are only reflected on the nanometer scale. The components of such coating agents suffer from issues such as poor biodegradability and easy environmental pollution caused by small-molecule surfactants, and their functions are relatively singular.

Based on this, an embodiment of the present disclosure is to provide a nanozyme, prepared from raw materials including, in parts by weight:
1 part to 6 parts of a metal salt;
0.5 parts to 4 parts of a surfactant; and
1 part to 1.04 parts of an acidic reagent; where
the metal salt includes one or more selected from the group consisting of an iron salt, a manganese salt, a calcium salt, a zinc salt, and a copper salt; and
an MOF material of the nanozyme is Prussian-blue.

It is understood that the research findings fully confirm that, as a typical MOF material, the crystal structure of Prussian-blue nanoparticles provides them with a stable skeleton, which enables them to maintain stable catalytic activity under various environmental conditions. Simultaneously, the surfaces are finely chemically modified, allowing them to effectively bind with target molecules (such as superoxide anions, hydrogen peroxide, and hydroxyl radicals, etc.), thereby accelerating their decomposition or transformation. They exhibit excellent antioxidant enzyme activities (primarily manifested as superoxide dismutase-like activity, catalase-like activity, and hydroxyl radical scavenging ability). This characteristic helps enhance crop resistance to abiotic stress. Furthermore, the metal elements doped in Prussian-blue derivative nanozymes have the effects of promoting crop, enhancing stress resistance, increasing yield, and improving quality of crops. Among them, manganese (Mn) can act as an activator for various enzymes in plants, participating in photosynthesis and respiration, regulating the ion balance within the plant, and improving the adaptability of plants to environmental stresses such as drought and salinity. Zinc (Zn) is an indispensable element for the growth and development of plants, participating in photosynthesis, respiration, nitrogen metabolism, hormone synthesis, and growth of plants. Zn promotes photosynthesis in crops, facilitates the accumulation of organic matter, and improves crop yield and stress resistance. Copper (Cu) participates in photosynthesis and respiration of plants, catalyzes redox reactions, promotes the metabolism and synthesis of carbohydrates and proteins, enhances the stability of chlorophyll, and improves the cold resistance and drought resistance of plants. Iron (Fe) is one of the essential trace elements in the process of the growth and development of plants, primarily promoting chlorophyll synthesis, participating in respiration, regulating the growth and development of plants, and enhancing stress resistance. Fe ions can catalyze the synthesis process of chlorophyll, giving plant leaves their green color, and participate in the transport and release of oxygen, as well as the catalytic activity of various enzymes. Calcium (Ca) is an essential trace element for certain lower plants and higher plants. In higher plants, Ca is the metal cofactor of urease, which is crucial for nitrogen metabolism. Ca also participates in the regulation of other enzyme activities, promoting the normal growth and development of plants.

In some embodiments of the present disclosure, the surfactant includes a tea seed cake extract.

In some embodiments of the present disclosure, the acidic reagent includes citric acid.

In the present disclosure, the raw materials for preparing the nanozyme include specific components with specific proportional relationships, where the metal salt provides an active center and a structural foundation of the nanozyme, that is, the presence of the metal ions can provide the active center, endowing the nanozyme with a catalytic activity, similar to the catalytic effect of the metal ions at an active site in natural enzymes; the surfactant controls the size and dispersity of the nanozyme; and the acidic reagent regulates reaction conditions. The coordination among these three raw materials provides a guarantee for the successful preparation and performance optimization of the nanozyme. Through the synergistic interaction of the components in their specific proportional relationships, a surfactant-modified nanomaterial exhibits desirable water solubility and can stably exist in the form of a high-concentration colloid. When being subsequently applied in seedling cultivation, the nanomaterial has the effects of promoting growth, enhancing stress resistance, increasing yield, and improving quality of crops.

Another embodiment of the present disclosure is to provide a method for preparing the nanozyme as described in the above solutions, including the following steps: mixing the raw materials for preparing the nanozyme, and subjecting a resulting mixture to a reaction and then drying to obtain the nanozyme.

In some embodiments of the present disclosure, the mixing is conducted at a stirring speed of 500 r/min to 700 r/min, and the mixing is conducted for 10 min to 30 min.

In some embodiments of the present disclosure, the reaction is conducted at a temperature of 60 °C to 80 °C, and the reaction is conducted for 24 h to 48 h.

In some embodiments of the present disclosure, the drying is conducted by a mode including one or more selected from the group consisting of vacuum drying and freeze drying.

In the present disclosure of the present disclosure, the method for preparing the nanozyme is characterized by simple processing and low cost. Using the nanozyme as a seed coating material can not only reduce the risk of phytotoxicity to seeds from traditional pesticides but also significantly improve the germination rate of seeds and the growth quality of seedlings.

Another embodiment of the present disclosure is to provide an agricultural formulation, including the nanozyme.

In some embodiments of the present disclosure, the agricultural formulation includes a seed coating agent; where an amount of the nanozyme is 5% to 9% of a total weight of the seed coating agent.

In some embodiment of the present disclosures, the seed coating agent includes following components, in parts by weight:
2 parts to 10 parts of a pesticide;
5 parts to 9 parts of the nanozyme;
1.5 parts to 3 parts of a thickener;
0.75 parts to 1.5 parts of a biobased polymer;
0.1 parts to 0.5 parts of an organic acid;
3 parts to 5 parts of an organic nitride;
0.5 parts to 1.5 parts of a mineral material; and
0.1 parts to 0.3 parts of a film-forming agent.

The aforementioned seed coating agent enables the nanozyme of the present disclosure to act in conjunction with the pesticide, the thickener, the biobased polymer, the organic acid, the organic nitride, the mineral material, and the film-forming agent. By controlling the specific proportions of the various preparation raw materials, the components act synergistically. The incorporation of the nanozyme enables both green and efficient disease control and provides nutrients for the crops, promoting the growth of the crops and enhancing the stress resistance of the crops. This gives the seed coating agent significant economic and ecological benefits, holding broad application prospects in the field of modern agriculture.

It should be noted that the value range of the pesticide is “2 parts to 10 parts”, meaning the minimum value and the maximum value of the range from 2 parts to 10 parts can be taken, as well as every value between this minimum and maximum. Specific examples include, but are not limited to, the point values in the examples and the following point values: 2 parts, 3 parts, 4 parts, 5 parts, 6 parts, 7 parts, 8 parts, 9 parts, 10 parts; or ranges formed by any two of these values, for example, including: 3 parts to 10 parts.

The value range of the nanozyme is “5 parts to 9 parts”, meaning the minimum value and the maximum value of the range from 5 parts to 9 parts can be taken, as well as every value between this minimum and maximum. Specific examples include, but are not limited to, the point values in the examples and the following point values: 5 parts, 6 parts, 7 parts, 8 parts, 9 parts; or ranges formed by any two of these values, for example, including: 6 parts to 9 parts.

The value range of the thickener is “1.5 parts to 3 parts”, meaning the minimum value and the maximum value of the range from 1.5 parts to 3 parts can be taken, as well as every value between this minimum and maximum. Specific examples include, but are not limited to, the point values in the examples and the following point values: 1.5 parts, 1.6 parts, 1.7 parts, 1.8 parts, 1.9 parts, 2.0 parts, 2.1 parts, 2.2 parts, 2.3 parts, 2.4 parts, 2.5 parts, 2.6 parts, 2.7 parts, 2.8 parts, 2.9 parts, 3.0 parts; or ranges formed by any two of these values, for example, including: 2 parts to 3 parts.

The value range of the biobased polymer is “0.75 parts to 1.5 parts”, meaning the minimum value and the maximum value of the range from 0.75 parts to 1.5 parts can be taken, as well as every value between this minimum and maximum. Specific examples include, but are not limited to, the point values in the examples and the following point values: 0.75 parts, 0.85 parts, 0.95 parts, 1.05 parts, 1.15 parts, 1.25 parts, 1.35 parts, 1.45 parts, or 1.5 parts; or ranges formed by any two of these values, for example, including: 1 part to 1.5 parts.

The value range of the organic acid is “0.1 parts to 0.5 parts”, meaning the minimum value and the maximum value of the range from 0.1 parts to 0.5 parts can be taken, as well as every value between this minimum and maximum. Specific examples include, but are not limited to, the point values in the examples and the following point values: 0.1 parts, 0.2 parts, 0.3 parts, 0.4 parts, 0.5 parts; or ranges formed by any two of these values, for example, including: 0.1 parts to 0.4 parts.

The value range of the organic nitride is “3 parts to 5 parts”, meaning the minimum value and the maximum value of the range from 3 parts to 5 parts can be taken, as well as every value between this minimum and maximum. Specific examples include, but are not limited to, the point values in the examples and the following point values: 3.0 parts, 3.1 parts, 3.2 parts, 3.3 parts, 3.4 parts, 3.5 parts, 3.6 parts, 3.7 parts, 3.8 parts, 3.9 parts, 4.0 parts, 4.1 parts, 4.2 parts, 4.3 parts, 4.4 parts, 4.5 parts, 4.6 parts, 4.7 parts, 4.8 parts, 4.9 parts, 5.0 parts; or ranges formed by any two of these values, for example, including: 3 parts to 4.5 parts.

The value range of the film-forming agent is “0.1 parts to 0.3 parts”, meaning the minimum value and the maximum value of the range from 0.1 parts to 0.3 parts can be taken, as well as every value between this minimum and maximum. Specific examples include, but are not limited to, the point values in the examples and the following point values: 0.1 parts, 0.2 parts, 0.3 parts; or ranges formed by any two of these values, for example, including: 0.1 parts to 0.2 parts.

In some embodiments of the present disclosure, the seed coating agent includes the following components, in parts by weight:
5 parts to 10 parts of the pesticide;
5 parts to 9 parts of the nanozyme;
1.5 parts to 2 parts of the thickener;
0.75 parts to 1 part of the biobased polymer;
0.1 parts to 0.25 parts of the organic acid;
3 parts to 5 parts of the organic nitride;
0.5 parts to 1 part of the mineral material; and
0.1 parts to 0.3 parts of the film-forming agent.

Furthermore, controlling the components of the seed coating agent within the aforementioned ratio ranges results in an even lower risk of phytotoxicity to seeds from traditional pesticides, as well as a superior germination rate of seeds and growth quality of seedlings.

In some embodiments of the present disclosure, the pesticide includes one or more selected from the group consisting of a fungicide and an insecticide.

In some embodiments of the present disclosure, the fungicide includes one or more selected from the group consisting of pyraclostrobin, iprodione, boscalid, and prothioconazole.

In some embodiments of the present disclosure, the insecticide includes one or more selected from the group consisting of cyhalothrin, emamectin benzoate, clothianidin, and thiamethoxam.

In some embodiments of the present disclosure, the thickener includes one or more selected from the group consisting of guar gum, pectin, gelatin, and starch.

In some embodiments of the present disclosure, the biobased polymer includes one or more selected from the group consisting of γ-polyglutamic acid, polylactic acid, polyhydroxyalkanoate, and chitin.

In some embodiments of the present disclosure, the organic acid includes selected from the group consisting one or more of salicylic acid, citric acid, ascorbic acid, and tartaric acid.

In some embodiments of the present disclosure, the organic nitride includes selected from the group consisting one or more of urea, an amino acid, polyacrylamide, and formamide.

In some embodiments of the present disclosure, the mineral material includes one or more selected from the group consisting of attapulgite, diatomite, bentonite, and sepiolite.

In some embodiments of the present disclosure, the film-forming agent includes one or more selected from the group consisting of polyvinyl alcohol, polyacrylate, chitosan, sodium carboxymethyl cellulose, a silicate, a natural plant oil, ethyl cellulose, sodium alginate, polyacrylic acid, starch, and lignin.

Another embodiment of the present disclosure is to provide a method for preparing the seed coating agent, including: mixing the components of the seed coating agent to obtain the seed coating agent.

Another embodiment of the present disclosure is to provide use of the agricultural formulation in improving a germination rate of a seed and a growth quality of a seedling.

In some embodiments of the present disclosure, the seed is derived from one or more selected from the group consisting of soybean, wheat, corn, rapeseed, peanut, cotton, cucumber, tomato, and carrot.

In the present disclosure, the agricultural formulation, through the highly efficient catalytic action of the nanozyme and the synergistic effects of the pesticide and fertilizer, possesses multiple advantages such as improving the pesticide and fertilizer efficacy, enhancing the disease resistance and stress resistance of seeds, reducing the usage of pesticides and chemical fertilizers, improving the soil environment, enhancing the quality of agricultural products, and enhancing the environmental protection advantages. This agricultural formulation effectively promotes the growth of crops and enhances the stress resistance of crops, giving it significant economic and ecological benefits and holding broad application prospects in the field of modern agriculture.

To make the objects, technical solutions, and advantages of the present disclosure more brief and clear, the present disclosure is illustrated with the following specific examples, but is by no means limited to these examples. The following examples are merely preferred examples of the present disclosure, can be used to describe the present disclosure, and should not be understood as limitations to the scope of the present disclosure. It should be pointed out that any modifications, equivalents, improvements, etc. made within the spirit and principle of the present disclosure shall all be included within the scope of the present disclosure.

To better explain the present disclosure, the present disclosure will be further explained below with reference to examples. In the following examples, unless otherwise specified, the experimental methods used are all conventional, and the reagents and materials used are commercially available.

### Example 1

(1) Preparation of a Prussian-blue derivative nanozyme: a nanozyme was composed of a metal salt, a surfactant, and an acidic reagent. The nanozyme was prepared by a specific process as follows: 1 part of the surfactant (tea seed cake extract), 1 part of the acidic reagent (citric acid), 2 parts of an iron salt (potassium ferrocyanide), and 0.9 parts each of a manganese salt (manganese chloride), a zinc salt (zinc nitrate), a copper salt (copper chloride), and a calcium salt (calcium chloride) were added to 50 mL of deionized water containing 0.02 parts of the acidic reagent. A resulting mixture was stirred at 600 r/min for 10 min at room temperature, placed in a vacuum drying oven, and subjected to a reaction at 80 °C for 20 h. A resulting product after the reaction was subjected to washing with deionized water, and then centrifugation with anhydrous ethanol. The washing and centrifugation process was repeated three times. A final product was freeze-dried to obtain a nanozyme powder. For details, please refer to Table 1.

(2) Preparation of a seed coating agent: in terms of parts by weight, 10 parts of fludioxonil and 0.1 parts of salicylic acid were mixed and ground until over 80% of resulting particles reached a size of 80 nm to 100 nm. Then, 3 parts of urea and 0.5 parts of attapulgite were added to the particles and mixed uniformly. Finally, 1.5 parts of guar gum, 0.75 parts of γ-polyglutamic acid, 5 parts of the nanozyme, and 0.3 parts of a film-forming agent (specifically, starch) were added thereto. A resulting mixture was stirred uniformly to obtain a seed coating agent.

(3) Preparation of a biofilm-based seed coating: soybean seeds were immersed in the seed coating agent for 1 min. After taking out, they were subjected to a drying treatment to obtain seeds with a coating.

### Examples 2 to 6

The preparation methods of the seed coating agents in Examples 2 to 6 were substantially consistent with that in Example 1, with the difference lying in the components of the seed coating agents. For details, please refer to Table 1 and Table 2.

The other steps and conditions were the same as those in Example 1.

### Example 7

The preparation method of the seed coating agent in Example 7 was substantially consistent with that in Example 1, with the difference lying in the proportion of the raw materials for preparing the nanozyme, specifically the proportions of the metal salt. In Example 7, the mixture consisted of 1 part of an iron salt (specifically potassium ferrocyanide), and 0.1 part each of a manganese salt (specifically manganese chloride), a zinc salt (specifically zinc nitrate), a copper salt (specifically copper chloride), and a calcium salt (specifically calcium chloride). For details, please refer to Table 1 and Table 2.

The other steps and conditions were the same as those in Example 1.

### Example 8

The preparation method of the seed coating agent in Example 8 was substantially consistent with that in Example 1, with the difference lying in the proportion of the raw materials for preparing the nanozyme, specifically the proportions of the metal salt. In Example 8, the mixture consisted of 2 parts of an iron salt (specifically potassium ferrocyanide), and 0.3 parts each of a manganese salt (specifically manganese chloride), a zinc salt (specifically zinc nitrate), a copper salt (specifically copper chloride), and a calcium salt (specifically calcium chloride). For details, please refer to Table 1 and Table 2.

The other steps and conditions were the same as those in Example 1.

### Comparative Example 1

The preparation method of the seed coating agent in Comparative Example 1 was substantially consistent with that in Example 1, with the difference lying in the proportion of the raw materials for preparing the nanozyme, specifically the proportion of the acidic reagent. In Comparative Example 1, 0.005 parts of a citric acid was used. For details, please refer to Table 1 and Table 2.

The other steps and conditions were the same as those in Example 1.

### Comparative Example 2

The preparation method of the seed coating agent in Comparative Example 2 was substantially consistent with that in Example 1, with the difference lying in the components of the seed coating agent. Specifically, the nanozyme was not introduced. For details, please refer to Table 2.

The other steps and conditions were the same as those in Example 1.

### Comparative Example 3

The preparation method of the seed coating agent in Comparative Example 3 was substantially consistent with that in Example 1, with the difference lying in the components of the seed coating agent, specifically the content of the organic acid (salicylic acid). Comparative Example 3 contained 0.6 parts of salicylic acid. For details, please refer to Table 1 and Table 2.

The other steps and conditions were the same as those in Example 1.

TABLE 1

| Component | Nanozyme 1 | Nanozyme 2 | Nanozyme 3 | Nanozyme 4 |
|---|---|---|---|---|
| Surfactant | 1 | 1 | 1 | 1 |
| Acidic reagent | 1.02 | 1.02 | 1.02 | 0.005 |
| Iron salt | 2 | 1 | 2 | 2 |
| Manganese salt | 0.9 | 0.1 | 0.3 | 0.9 |
| Zinc salt | 0.9 | 0.1 | 0.3 | 0.9 |
| Copper salt | 0.9 | 0.1 | 0.3 | 0.9 |
| Calcium salt | 0.9 | 0.1 | 0.3 | 0.9 |

TABLE 2

| | | | | | | | | | Compar- | Compar- | Compar- |
|---|---|---|---|---|---|---|---|---|---|---|---|
| | | | | | | | | | ative | ative | ative |
| | Exam- | Exam- | Exam- | Exam- | Exam- | Exam- | Exam- | Exam- | Exam- | Exam- | Exam- |
| Component | ple 1 | ple 2 | ple 3 | ple 4 | ple 5 | ple 6 | ple 7 | ple 8 | ple 1 | ple 2 | ple 3 |
| Nanozyme 1 | 5 | 7 | 9 | 5 | 7 | 9 | / | / | / | / | 5 |
| Nanozyme 2 | / | / | / | / | / | / | 5 | / | / | / | / |
| Nanozyme 3 | / | / | / | / | / | / | / | 5 | / | / | / |
| Nanozyme 4 | / | / | / | 1 | / | / | | / | 5 | / | / |
| Fludioxonil | 10 | 5 | 2 | 10 | 5 | 2 | 10 | 10 | 10 | 10 | 10 |
| Salicylic | 0.1 | 0.25 | 0.5 | 0.1 | 0.25 | 0.5 | 0.1 | 0.1 | 0.1 | 0.1 | 0.6 |
| acid | | | | | | | | | | | |
| Urea | 3 | 4 | 5 | 5 | 3 | 4 | 3 | 3 | 3 | 3 | 2 |
| Attapulgite | 0.5 | 1 | 1.5 | 0.5 | 1 | 1.5 | 0.5 | 0.5 | 0.5 | 0.5 | 0.5 |
| Guar | 1.5 | 2 | 3 | 1.5 | 2 | 3 | 1.5 | 1.5 | 1.5 | 1.5 | 3 |
| gum | | | | | | | | | | | |
| γ- | 0.75 | 1 | 1.5 | 0.75 | 1 | 1.5 | 0.75 | 0.75 | 0.75 | 0.75 | 0.35 |
| polyglutamic | | | | | | | | | | | |
| acid | | | | | | | | | | | |
| Film- | 0.3 | 0.1 | 0.1 | 0.1 | 0.2 | 0.2 | 0.3 | 0.3 | 0.3 | 0.3 | 0.3 |
| forming | | | | | | | | | | | |
| agent | | | | | | | | | | | |

### Test

### 1. Film-Forming Time Detection for Different Formulations

### (1) Experimental Method

When a desirable suspension seed coating agent coats seeds, a liquid medicine can rapidly solidify and form a film on a surface of the seeds, creating a protective layer against diseases and pests. Film-forming property is an important detection index during the application of suspension seed coating agents. Detection method: 50 g of seeds (weighed precisely to 1 g) were placed in a petri dish. 1 g of the sample was drawn using a syringe and injected into the petri dish. The dish was covered with a lid and inverted for 5 min. Subsequently, the lid was opened, and resulting coated seeds were spread out flat to allow film formation. Resulting seeds were gently stirred with a glass rod, and seed surfaces were observed. If the seed coating agent on all seed surfaces had solidified into a film, the film-forming performance was considered qualified. The film-forming time for each formulation was recorded separately.

### (2) Data Analysis

Test results are shown in Table 3. It is clearly observed that the formulation of Example 1 exhibits rapid film-forming performance.

TABLE 3

| Film- | | | | | | | | | | | |
|---|---|---|---|---|---|---|---|---|---|---|---|
| forming | | | | | | | | | | | |
| time | | | | | | | | | Comparative | Comparative | Comparative |
| (min) | Example 1 | Example 2 | Example 3 | Example 4 | Example 5 | Example 6 | Example 7 | Example 8 | Example 1 | Example 2 | Example 3 |
| 1 | 16 | 28 | 28 | 29 | 21 | 19 | 21 | 25 | 21 | 23 | 29 |
| 2 | 14 | 25 | 29 | 31 | 22 | 20 | 22 | 26 | 20 | 25 | 18 |
| 3 | 15 | 22 | 30 | 33 | 23 | 19 | 23 | 27 | 22 | 24 | 17 |
| Mean | 15 | 25 | 29 | 31 | 22 | 19 | 22 | 26 | 21 | 24 | 28 |

### 2. Coating Uniformity Detection for Different Formulations

### (1) Experimental Method

From the coated seeds prepared using different formulations, 100 coated seeds that had passed the film-forming qualification test were randomly selected. These seeds were placed into 25 stoppered centrifuge tubes (4 seeds per tube), respectively. Using a pipette, 2.0 mL to 5.0 mL of an ethanol solution was accurately added to each tube (ensuring absorbance falls within a linear range). The centrifuge tubes were capped, and the seeds were soaked for 1 h, then shaken for extraction for 15 min. After standing or centrifugation, a clear solution was obtained. Using the ethanol solution as a reference, an absorbance (A) of the extraction solution was measured at its maximum absorption wavelength. 25 measured absorbance values were arranged in ascending order, and an average absorbance value A was calculated. A coating uniformity X (%) was calculated as X (%)=n/25×100%, where n represents a number of centrifuge tubes whose measured absorbance A falls within the range of 0.7A to 1.3A, and 25 represents a total number of centrifuge tubes.

### (2) Data Analysis

Test results are shown in Table 4. It is clearly seen that the coating effect of the formulation of Example 1 is the most uniform.

TABLE 4

| Coating | | | | | | | | | | |
|---|---|---|---|---|---|---|---|---|---|---|
| uniformity | | | | | | | | Comparative | Comparative | Comparative |
| (%) | Example 1 | Example 2 | Example 3 | Example 4 | Example 5 | Example 6 | Example 7 | Example 1 | Example 2 | Example 3 |
| 1 | 98 | 95 | 96 | 93 | 92 | 96 | 96 | 96 | 95 | 92 |
| 2 | 96 | 94 | 95 | 92 | 94 | 97 | 95 | 94 | 97 | 94 |
| 3 | 97 | 93 | 94 | 94 | 96 | 96 | 94 | 92 | 96 | 96 |
| Mean | 97 | 94 | 95 | 93 | 94 | 96 | 95 | 94 | 96 | 94 |

### 3. Detachment Rate Detection for Different Formulations

### (1) Experimental Method

Two portions of coated seeds, each weighing 10 g (weighed precisely to 0.02 g) that had passed the film-forming test, were weighed and placed separately into conical flasks. To the first portion, 100 mL of ethanol was accurately added thereto. The flask was stoppered and placed in an ultrasonic cleaner, where it was oscillated for 10 min to fully dissolve the seed coating agent on the seed surfaces. The flask was then removed and allowed to stand for 10 min. Subsequently, 5 mL of this solution was transferred from the flask to a 50 mL volumetric flask, diluted to a mark with ethanol, and mixed thoroughly to obtain Solution A. The second portion of coated seeds was placed in a shaker and oscillated for 10 min. Afterwards, the seeds were carefully transferred from the shaker to another conical flask. Following the same treatment procedure used for Solution A, Solution B was prepared. Using ethanol as a reference, an absorbance of the solutions was measured at their maximum absorption wavelength. A post-coating detachment rate X (%) was calculated as: X (%)=(A₀m₁−A₁m₀)/A₀m₁×100%, where m₀ represents a mass (in g) of the coated seeds weighed for preparing Solution A; m₁ represents a mass (in g) of the coated seeds weighed for preparing Solution B; A₀ represents an absorbance of Solution A; and A₁ represents an absorbance of Solution B.

### (2) Data Analysis

Test results are shown in Table 5. It is clearly observed that the formulation of Example 1 exhibits excellent anti-detachment performance.

TABLE 5

| Shedding | | | | | | | | | Comparative | Comparative | Comparative |
|---|---|---|---|---|---|---|---|---|---|---|---|
| rate (%) | Example 1 | Example 2 | Example 3 | Example 4 | Example 5 | Example 6 | Example 7 | Example 8 | Example 1 | Example 2 | Example 3 |
| 1 | 4.5 | 5.1 | 5.7 | 4.9 | 5 | 4.9 | 6.2 | 5.8 | 6.0 | 6.1 | 5.2 |
| 2 | 4.2 | 5.9 | 5.5 | 5.7 | 5.2 | 5 | 6.4 | 5.7 | 5.8 | 6.2 | 5.4 |
| 3 | 4.1 | 6 | 4.9 | 5.4 | 5.3 | 5.3 | 6.0 | 5.9 | 5.9 | 6.3 | 5.6 |
| Mean | 4.3 | 5.7 | 5.4 | 5.3 | 5.2 | 5.1 | 6.3 | 5.8 | 5.9 | 6.3 | 5.6 |

### 4. Phytotoxicity Detection of Different Formulations on Soybeans

### (1) Experimental Method

Coated seeds from the examples and comparative examples were placed in 9 cm petri dishes, with 10 seeds per dish. Each treatment group was set up with 3 repetitions. The seeds were cultivated in an environment maintained at 60% humidity. A germination rate was counted after three days. Uncoated seeds were used as a blank control.

### (2) Data Analysis

Test results are shown in Table 6. It is clearly seen that the formulation of Example 1 does not cause phytotoxicity to the soybeans.

TABLE 6

| | | | | | | | | | | Compar- | Compar- | Compar- |
|---|---|---|---|---|---|---|---|---|---|---|---|---|
| | | | | | | | | | | ative | ative | ative |
| Germination | | Exam- | Exam- | Exam- | Exam- | Exam- | Exam- | Exam- | Exam- | Exam- | Exam- | Exam- |
| rate (%) | Control | ple 1 | ple 2 | ple 3 | ple 4 | ple 5 | ple 6 | ple 7 | ple 8 | ple 1 | ple 2 | ple 3 |
| 1 | 100 | 90 | 90 | 100 | 90 | 90 | 100 | 96 | 92 | 94 | 90 | 96 |
| 2 | 90 | 100 | 90 | 90 | 100 | 90 | 90 | 94 | 93 | 92 | 94 | 92 |
| 3 | 100 | 100 | 100 | 100 | 100 | 100 | 90 | 92 | 94 | 90 | 92 | 90 |
| Mean | 96.7 | 96.7 | 93.3 | 96.7 | 96.7 | 93.3 | 93.3 | 94 | 93 | 92 | 92 | 92 |

### 5. Detection of Control Efficacy of Different Formulations Against Soybean Root Rot

### (1) Experimental Method

Pythium aphanidermatum was compounded into soil to form infested soil, and the infested soil was potted. The coated seeds (FIG. 3) were then sown into the pots, with 10 seeds per pot. Each treatment group consisted of 4 pots. When the seedlings reached the 3-leaf stage, the pots were overturned to investigate the disease incidence rate and disease rating. A control efficacy was calculated. Uncoated seeds were used as a blank control.

### (2) Data Analysis

Test results are shown in Table 7. It is clearly observed that the formulation of Example 1 exhibits excellent control efficacy against soybean root rot.

TABLE 7

| | | | | | | | | | Compar- | Compar- | Compar- |
|---|---|---|---|---|---|---|---|---|---|---|---|
| | | | | | | | | | ative | ative | ative |
| Efficacy | Exam- | Exam- | Exam- | Exam- | Exam- | Exam- | Exam- | Exam- | Exam- | Exam- | Exam- |
| (%) | ple 1 | ple 2 | ple 3 | ple 4 | ple 5 | ple 6 | ple 7 | ple 8 | ple 1 | ple 2 | ple 3 |
| 1 | 87.4 | 55.1 | 21.7 | 84.9 | 55 | 24.9 | 80.1 | 76.9 | 19.3 | 21.4 | 21.9 |
| 2 | 83.1 | 55.9 | 22 | 85.7 | 45.2 | 25 | 79.2 | 75.3 | 21.2 | 19.9 | 22.7 |
| 3 | 80.9 | 66 | 25.9 | 85.4 | 65.3 | 25.3 | 78.4 | 81.2 | 20.1 | 18.7 | 23.1 |
| Mean | 83.8 | 59 | 23.2 | 85.3 | 55.2 | 25.1 | 79.23 | 77.80 | 20.2 | 20.00 | 25.57 |

### 6. Effect of the Seed Coating Agent in Example 1 on Soybean Germination Rate and Sprout Length

### (1) Experimental Method

Coated seeds prepared in Example 1 were placed on a filter paper, with 25 seeds per group and a total of 3 groups. Uncoated seeds were used as a blank control. Humidity was maintained at 60% to 90%. A germination rate was counted, and sprout length was measured after 3 d.

### (2) Data Analysis

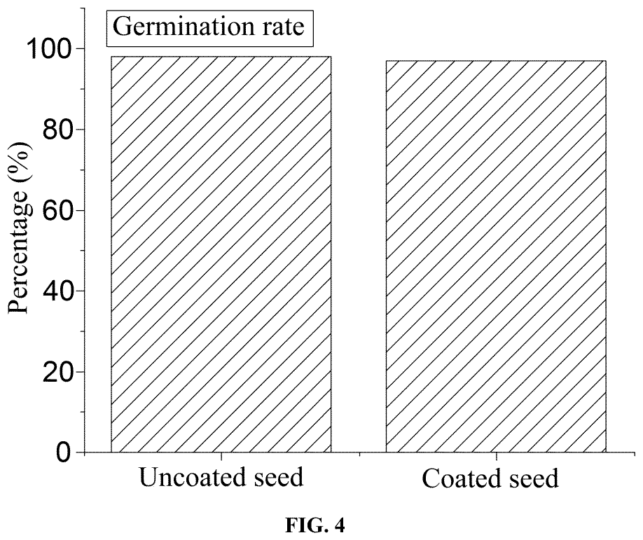

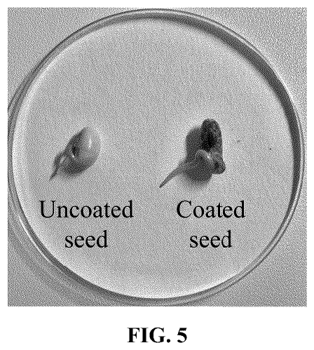

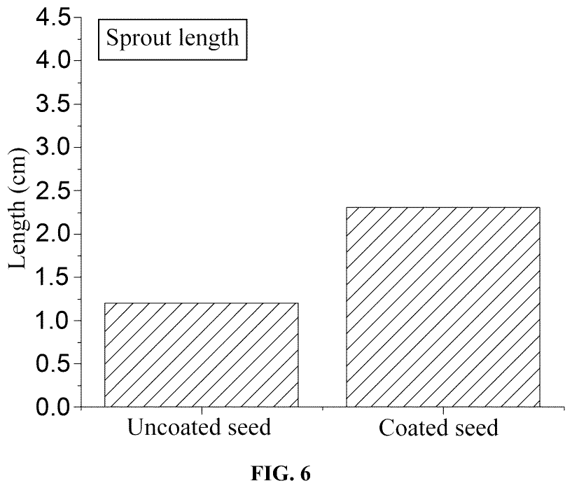

Test results are shown in FIG. 4 and FIG. 5. It is clearly observed that coating does not adversely affect soybean germination, as there is no significant difference in germination rate. However, the coating significantly promotes the sprout length growth (FIG. 6).

### 7. Effect of the Seed Coating Agent in Example 1 on Soybean Growth

### (1) Experimental Method

Coated seeds were sown in soil, with uncoated seeds using as a control group. The potted plants were cultivated in a light incubator. After 1 week, a root length, a plant height, and a fresh weight of each plant were observed and recorded.

### (2) Data Analysis

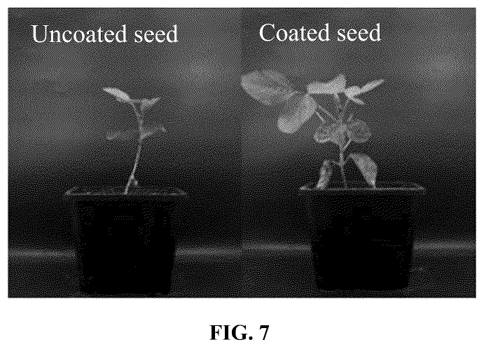

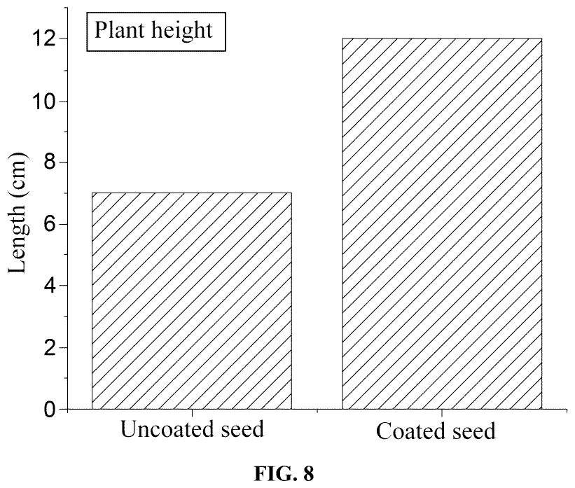

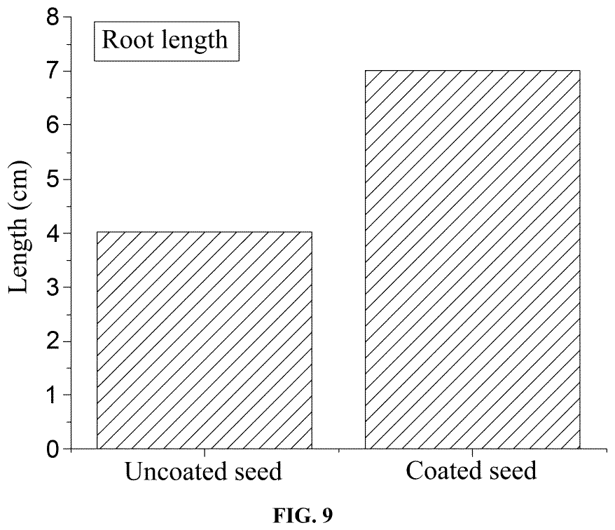

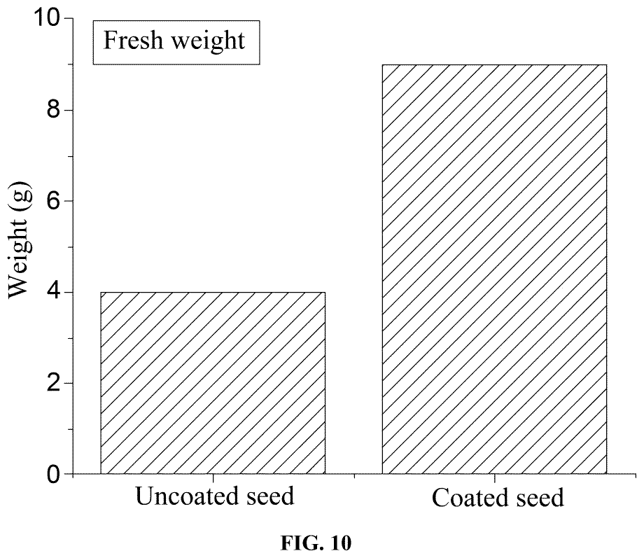

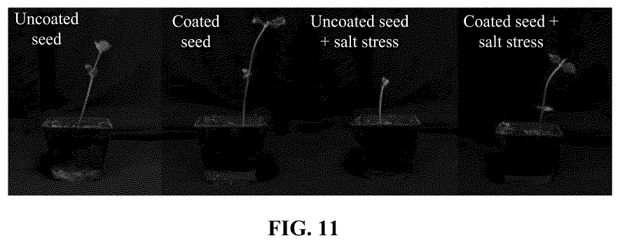

Test results are shown in FIG. 7. It is seen that the seed coating agent in Example 1 promotes the growth of soybean, specifically manifested in the promotion of the root length, the plant height, and the fresh weight (FIG. 8, FIG. 9, and FIG. 10).

### 8. Enhancement of Plant Salt Stress Resistance by the Seed Coating Agent in Example 1

### (1) Experimental Method

Coated seeds were sown in soil, with uncoated seeds using as a control group. The potted plants were cultivated in a light incubator. One group of coated seeds and one group of uncoated seeds were treated with a 0.1% NaCl solution to simulate salt stress conditions, respectively. After 1 week, a growth status of each plant was observed and recorded, and a leaf chlorophyll content was measured.

### (2) Data Analysis

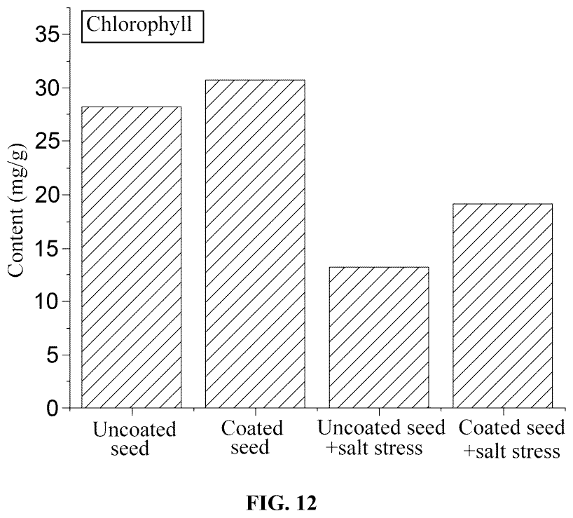

Test results are shown in FIG. 11. It is seen that the seed coating agent in Example 1 enhances the salt stress resistance of soybeans, and the chlorophyll content is also higher than that of uncoated seeds (FIG. 12).

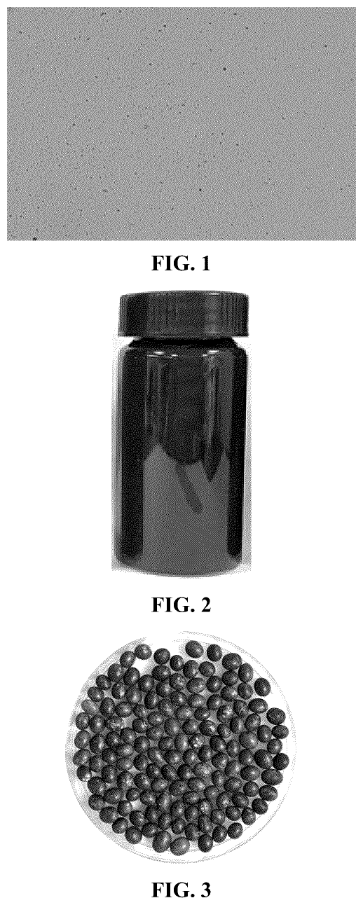

Based on the results from Tables 3 to 7 and FIG. 1 to FIG. 12: FIG. 1 shows an optical microscope image of the agricultural formulation in Example 1; FIG. 2 shows a photograph of the agricultural formulation in Example 1; FIG. 3 shows a phenotypic image of the agricultural formulation in Example 1; FIG. 4 shows a histogram of the effect of the agricultural formulation in Example 1 on soybean germination rate; FIG. 5 shows an effect diagram of the agricultural formulation in Example 1 on soybean germination status; FIG. 6 shows a histogram of the effect of the agricultural formulation in Example 1 on soybean sprout length; FIG. 7 shows a photograph of the promotion of soybean growth by the agricultural formulation in Example 1; FIG. 8 shows the effect of the agricultural formulation in Example 1 on soybean plant height; FIG. 9 shows the effect of the agricultural formulation in Example 1 on soybean root length; FIG. 10 shows the effect of the agricultural formulation in Example 1 on the fresh weight of soybean plants; FIG. 11 shows a photograph of the effect of the agricultural formulation in Example 1 on salt stress resistance in soybean plants; and FIG. 12 shows a histogram of chlorophyll content in soybean plants after salt stress under treatment with the agricultural formulation in Example 1.

The agricultural formulation demonstrates the following effects: 1. Resolution of Phytotoxicity Issues: as an active oxygen catalytic materials, the nanozyme offers the advantages of high efficiency, low toxicity, and repeatable catalysis, which can effectively degrade free radicals produced under pesticide stress, reducing their toxic effects on biological systems. In the agricultural field, the application of nanozymes not only reduces the risk of phytotoxicity to seeds from traditional pesticides but also significantly improves the germination rate of seeds and the growth quality of seedlings. This technology provides a safer and more environmentally friendly solution for agricultural production, being helpful to enhance the yield and quality of crops. 2. Promotion of Crop Growth: through the application of nanotechnology, the pesticide and fertilizer components can be more effectively attached to the seed surface, forming a durable protective layer that continuously inhibits the invasion and spread of pests and diseases while simultaneously providing essential nutritional support. Leveraging their small size and large specific surface area, nanozymes can interact with the seed surface more efficiently, significantly improving the utilization rate of pesticide and fertilizer efficiency. Furthermore, the in-situ catalytic oxygen generation property of the nanozyme provides sufficient oxygen for rhizosphere microorganisms, further promoting the healthy growth of crops. This innovative technology offers a more efficient and environmentally friendly solution for agricultural production, enhancing the yield of crops. 3. Enhancement of Seed Disease Resistance: the nanozyme coating effectively protects plants either by directly destroying pathogens or by enhancing the plant's own immune system (e.g., by increasing the antioxidant enzyme activity of crops). This technology can significantly improve the disease resistance of seeds, reduce the risk of crops being attacked by diseases and insect pests, thereby increasing the yield and quality of crops. The nanozyme coating provides an efficient and sustainable solution for plant disease control, with broad application prospects. 4. Improvement of Stress resistance: the pesticide components in the seed coating agent effectively prevent and control pests and diseases, while the introduction of nanozyme exerts multiple enzyme-like activities, efficiently catalyzing active oxygen, thus further enhancing the stress resistance of crops, whether in response to biotic or abiotic stress. The unique structure of the nanozyme also enhances the adaptability of crops to adverse environments, providing a more comprehensive protective mechanism for crops. This technology not only improves the stress resistance of crops but also reduces the threat of pests and diseases, achieving the synergistic effect between pesticides and fertilizers and optimizing agricultural production effects. 5. Reduction of Pesticide Usage: due to their high specific surface area and porosity, nanozymes are helpful to achieve the slow-release effect for pesticide active ingredients, thereby significantly improving the utilization rate of pesticides. Simultaneously, nanozymes can improve the dispersibility and stability of pesticides, enhance their solubility and mobility in soil, and promote the effective utilization of pesticides by plants. Through this mechanism, not only the amount of pesticide used is reduced, but also the risk of environmental pollution is lowered, achieving the purpose of more environmentally friendly and efficient agricultural production. 6. Increase in the Yield and Quality of Agricultural Product: the agricultural formulation in the embodiments of the present disclosure significantly enhances the yield and quality of crops by promoting crop growth and enhancing stress resistance. Furthermore, due to the effective reduction in pesticide usage, the safety of agricultural products is improved, thereby enhancing their market competitiveness. This innovative technology not only optimizes agricultural production efficiency but also provides strong support for sustainable agricultural development. 7. Environmental Protection and Cost Advantages: nanozyme coating technology offers environmental protection advantages, which can reduce the loss and volatilization of pesticides, thereby reducing environmental pollution. At the same time, this technology can also improve the environmental behavior and biosafety of pesticides, enhancing the safety of chemical control for the ecological environment. The raw materials for this seed coating have a wide source and a simple preparation process, and are easy for large-scale batch production.

In summary, through the highly efficient catalytic action of the nanozyme and the synergistic effects of the pesticide and fertilizer, the agricultural formulation possesses multiple advantages such as improving the pesticide and fertilizer efficacy, enhancing the disease resistance and stress resistance of seeds, reducing the usage of pesticides and chemical fertilizers, improving the soil environment, enhancing the quality of agricultural products, and enhancing the environmental protection advantages. This agricultural formulation effectively promotes the growth of crops and enhances the stress resistance of crops, giving the agricultural formulation significant economic and ecological benefits and holding broad application prospects in the field of modern agriculture.

The technical features of the foregoing embodiments can be combined arbitrarily. For brevity of description, not all possible combinations of the technical features in the foregoing embodiments are described. However, the combinations of the technical features should be construed as the scope described in this specification as long as there is no contradiction between the combinations.

The foregoing examples only express several embodiments of the present disclosure, and their descriptions are more specific and detailed, but they should not therefore be construed as limiting the scope of the present disclosure. It should be noted that those of ordinary skill in the art can further make several variations and improvements without departing from the conception of the present disclosure. These variations and improvements all fall within the scope of the present disclosure. Therefore, the scope of the present disclosure should be subject to the scope of the appended claims.

What is claimed is:

1. An agricultural formulation, being selected from a seed coating agent, wherein the agricultural formulation comprises following components, in parts by weight:
5 parts to 10 parts of a pesticide;
5 parts to 9 parts of a nanozyme;
1.5 parts to 3 parts of a thickener;
0.75 parts to 1.5 parts of a biobased polymer;
0.1 parts to 0.5 parts of an organic acid;
3 parts to 5 parts of an organic nitride;
0.5 parts to 1.5 parts of a mineral material; and
0.1 parts to 0.3 parts of a film-forming agent;
wherein the pesticide is selected from fludioxonil; the organic acid is selected from salicylic acid; the mineral material is selected from attapulgite; the thickener is selected from guar gum; the organic nitride is selected from urea; the biobased polymer is selected from γ-polyglutamic acid; the film-forming agent is selected from starch; and
the nanozyme is prepared from raw materials comprising, in parts by weight:
3.2 part to 5.6 parts of a metal salt;
0.5 parts to 4 parts of a surfactant; and
1 part to 1.02 parts of an acidic reagent;
wherein the metal salt comprises potassium ferrocyanide, as well as manganese chloride, zinc nitrate, copper chloride, and calcium chloride; the surfactant is selected from a tea seed cake extract; and the acidic reagent is selected from citric acid.

2. The agricultural formulation of claim 1, wherein the nanozyme is prepared by a method comprising:
mixing the raw materials for preparing the nanozyme, and subjecting a resulting mixture to a reaction and then drying to obtain the nanozyme.

3. The agricultural formulation of claim 2, wherein the mixing is conducted at a stirring speed of 500 r/min to 700 r/min for 10 min to 30 min;
the reaction is conducted at a temperature of 60 °C to 80 °C for 24 h to 48 h; and
the drying is conducted by a mode comprising one or more selected from the group consisting of vacuum drying and freeze drying.

4. Use of the agricultural formulation of claim 1 in improving a germination rate of a seed and a growth quality of a seedling.

5. The use of claim 4, wherein the seed is derived from one or more selected from the group consisting of soybean, wheat, corn, rapeseed, peanut, cotton, cucumber, tomato, and carrot.

6. The use of claim 4, wherein the nanozyme is prepared by a method comprising:
mixing the raw materials for preparing the nanozyme, and subjecting a resulting mixture to a reaction and then drying to obtain the nanozyme.

7. The use of claim 4, wherein the mixing is conducted at a stirring speed of 500 r/min to 700 r/min for 10 min to 30 min;
the reaction is conducted at a temperature of 60 °C to 80 °C for 24 h to 48 h; and
the drying is conducted by a mode comprising one or more selected from the group consisting of vacuum drying and freeze drying.
